(19) United States
(12) Patent Application Publication
(10) Pub. No.: US 2024/0124831 A1
(43) Pub. Date: Apr. 18, 2024

(54) MICROORGANISM-DERIVED MATERIAL AND METHODS FOR PRODUCING SAME

(71) Applicant: YEAP LTD., Ashdod (IL)

(72) Inventors: Didier TOUBIA, Raanana (IL); Dominik GRABINSKI, San Francisco, CA (US); Keren KLES, Ness Ziona (IL); Yonatan GOSHEN, Ramat Gan (IL)

(73) Assignee: YEAP LTD., Ashdod (IL)

(21) Appl. No.: 18/278,242

(22) PCT Filed: Feb. 22, 2022
(86) PCT No.: PCT/IL2022/050210

§ 371 Date: Aug. 22, 2023

Related U.S. Application Data
(60) Provisional application No. 63/152,142, filed on Feb. 22, 2021.

(51) Int. Cl. C12N 1/06; A23J 1/18; A23J 3/20

(52) U.S. Cl. CPC C12N 1/063; A23J 1/18; A23J 3/20; C12N 1/066; C12R 2001/465

(57) ABSTRACT

Disclosed herein is a method for separating a protein from a microorganism comprising a cell wall. Further compositions comprising a first fraction, a second fraction or both, derived from a microorganism comprising a cell wall and comprising a protein content between 10% and 90% by weight of the fraction, are also disclosed.

### CROSS REFERENCE TO RELATED APPLICATIONS

This application claims the benefit of priority to U.S. Provisional Patent Application No. 63/152,142 filed Feb. 22, 2021 entitled “MICROORGANISM-DERIVED MATERIAL AND METHODS FOR PRODUCING SAME”, the contents of which are incorporated herein by reference in their entirety.

### FIELD OF THE INVENTION

The present invention is directed to processes for separation of microorganism-derived materials, including proteins from bacteria, fungi and microalgae, and compositions comprising microorganism-derived materials and proteins derived from bacteria, fungi and/or microalgae.

### BACKGROUND OF THE INVENTION

As the human population continues to increase, there's a growing need for additional food sources alternatively to meat and dairy sources, particularly food sources that are inexpensive to produce but provide a good nutritional value. Moreover, meat production, contributes significantly to the release of greenhouse gases, and there's a need for new foodstuffs that are equally tasty and nutritious yet less harmful to the environment to produce.

Consumers are increasingly looking for quality, natural ingredients and authenticity. However, meat substitutes are seen as highly processed products. There is still a great need for food product alternatives that can provide the taste, texture, and sensorial experience of traditional animal products, while using an affordable, widely available sources that are naturally rich in proteins. Moreover, there is still a great need for efficient and simple procedures for processing and isolating single-cell proteins from microorganisms, to be used in meat-alternative products.

### SUMMARY OF THE INVENTION

In one aspect of the invention, there is provided a method for A method for optimizing a protein content extracted from a microorganism comprising a cell wall, the method comprising: a) breaking the cell wall of the microorganism to a particle size suitable for enzymatic reaction; and b) receiving a first fraction comprising soluble proteins and a second fraction comprising insoluble proteins, wherein any one of the first and the second fraction comprises a protein content between 10% and 90% by weight of the fraction, thereby optimizing the protein content extracted from the microorganism comprising a cell wall.

In some embodiments, the particle size suitable for enzymatic reaction is between 2 μm and 10 μm.

In some embodiments, breaking the cell wall comprises applying any one of a) pressure between 150 bars and 1500 bars; b) temperature between 50° C. and 100° C.; c) ultrasonication, d) bead mill homogenization, or any combination thereof to the microorganism.

In some embodiments, the first fraction, the second fraction, or both is suitable for extrusion.

In some embodiments, the microorganism is a bacteria, a fungi or a microalgae.

In some embodiments, the fungi is a yeast.

In some embodiments, the yeast comprises Saccharomyces cerevisiae, Streptomyces natalensis, Streptomyces chattanoogensis, Saccharomyces fragilis, Candida utilis, Candida guilliermondii, Candida lipolityca, Cyberlindnera jadinii, Pichia pastoris, or any combination thereof.

In some embodiments, the yeast is derived from downstream food-related industries.

In some embodiments, the yeast comprises spent yeast, instant yeast, inactive yeast, live yeast, or any combination thereof.

In some embodiments, the first fraction and second fraction comprises a particle characterized by an average particle size between 1 μm and 100 μm.

In some embodiments, the protein is characterized by a molecular weight between 1 kDa and 250 kDa.

In some embodiments, the insoluble protein is characterized by an aqueous solubility of less than 300 g/L.

In some embodiments, the applying pressure comprises high-pressure homogenization.

In some embodiments, the method further comprises a step comprising removing a fiber from the cell wall or portion thereof.

In some embodiments, removing a fiber comprises chemical hydrolysis, enzymatic hydrolysis, or both.

In some embodiments, the enzymatic hydrolysis comprises contacting the microorganism with an enzyme, wherein the enzyme is selected from the group consisting of: lipase, phospolipase, polygalacturonase, chitinase, pectinase, β-glucanase, protease, arabinanase, pectinase, endo-amalyase, endo-β-glycanase, hydrolase, cellulose, arabanase, cellulase, hemicellulase, xylanase, laccase, mannanase, exo-endo-protease, or any combination thereof.

In some embodiments, the enzyme comprises phospolipase, polygalacturonase, chitinase, pectinase, β-glucanase, protease and arabinanase.

In some embodiments, the enzymatic hydrolysis is performed at a pH between 3.5 and 7.

In some embodiments, the method comprises repeating the applying pressure between 150 bars and 1500 bars.

In some embodiments, the method further comprises a step comprising cleaning, grinding, milling, washing, drying or any combination thereof, the microorganism.

In some embodiments, any one of the first fraction and the second fraction is in the form of a powder, solution, suspension, or any combination thereof.

In some embodiments, the method further comprises a step comprising isolating, purifying, concentrating, or any combination thereof, a protein from the first fraction, the second fraction or both.

In one aspect of the invention, there is provided a composition comprising a first fraction, a second fraction or both, derived from a microorganism comprising a cell wall and comprising a protein content between 10% and 90% by weight of the fraction, obtained by the method of the present invention.

In one aspect of the invention, there is provided a composition comprising more than 50% by dry weight of a protein derived from a microorganism comprising a cell wall.

In some embodiments, the protein is characterized by a molecular weight between 1 kDa and 250 kDa.

In some embodiments, the composition is characterized by an aqueous solubility of less than 300 g/L.

In some embodiments, the protein is characterized by an amino acid profile presented in Table 13.

In some embodiments, the composition is in the form of a powder characterized by an average particle size between 1 μm and 100 μm.

In some embodiments, the composition comprises a fiber content between 5% and 20% by dry weight.

In some embodiments, the composition comprises an ash content of less than 5%.

In some embodiments, the composition is characterized by a protein digestibility-corrected amino acid score (PDCAAS) between 0.7 and 1.

In some embodiments, the microorganism is a bacteria, a fungi or a microalgae.

In some embodiments, the fungi is a yeast.

In some embodiments, the yeast comprises Saccharomyces cerevisiae, Streptomyces natalensis, Streptomyces chattanoogensis, Saccharomyces fragilis, Candida utilis, Candida guilliermondii, Candida lipolityca, Cyberlindnera jadinii, Pichia pastoris, or any combination thereof.

In some embodiments, the yeast is derived from downstream food-related industries.

In some embodiments, the yeast comprises spent yeast, instant yeast, inactive yeast, live yeast, or any combination thereof.

In one aspect of the invention, there is provided a food product comprising the composition of the present invention.

In some embodiments, the food product is characterized as being suitable for use as an equivalent product to meat, eggs, cheese, dairy products, meat substitute products, plant-based products, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-B include bar graphs of progression of protein content (over DM) as function of high-pressure homogenization experiments (FIG. 1A) and progression of protein content (over DM) as function of hydrolysis and homogenization experiments (FIG. 1).

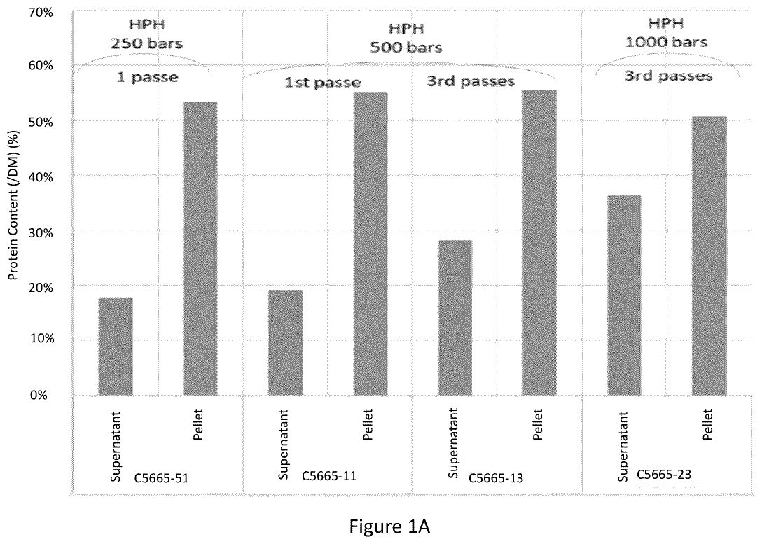

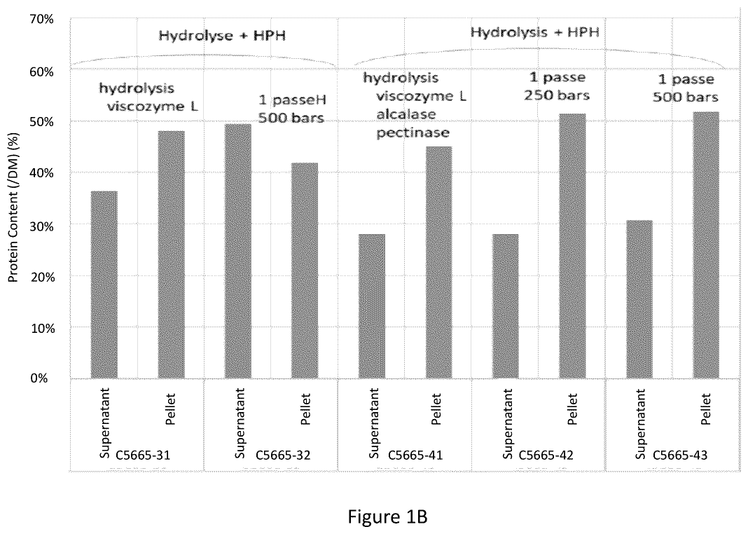

### DETAILED DESCRIPTION OF THE INVENTION

The present invention is directed to a method for optimizing a protein content extracted from a microorganism comprising a cell wall. The present invention is directed to a method for separating a protein from a microorganism comprising a cell wall, by breaking the cell wall of the microorganism to a particle size suitable for enzymatic reaction. The present invention is directed to a method for separating a protein from a microorganism comprising a cell wall, by receiving a first fraction comprising soluble proteins and a second fraction comprising insoluble proteins, wherein any one of the first and the second fraction comprises a protein content between 10% and 90% by weight of the fraction.

In some embodiments, the microorganism is a bacteria, a fungi or a microalgae. In some embodiments, the fungi is a yeast.

In some embodiments there is provided a method for obtaining a yeast material from yeast comprising a protein content between 10% and 90% by weight of the yeast material.

The present invention is based, in part, on the finding that any one of the first fraction and the second fraction comprising a protein content between 10% and 90% by weight of the fraction, are suitable for extrusion.

The present invention is based, in part, on the finding that using specific conditions to break the cell wall of the microorganism to a particle size suitable for enzymatic reaction, provides an increase in the final protein content separated from the microorganism. In some embodiments, the conditions provided herein, provide a composition with increased protein content and purity. In some embodiments, the conditions provided herein, provide a composition with increased emulsification capacity. As used herein “emulsification capacity” refers to the capacity of the composition to form a substantially stable emulsion. In some embodiments, the term “stable” refers to the ability of the composition to substantially maintain its structural, physical, and/or chemical properties. In some embodiments, the term “stable” refers to an emulsion devoid of phase separation, precipitation and/or decomposition. In some embodiments, the emulsion is an oil in water emulsion. In some embodiments, a composition described herein, is capable of forming an emulsion characterized by a small droplet size. In some embodiments, the droplets are stable over time.

The present invention is also directed to a composition comprising a first fraction, a second fraction or both, derived from a microorganism comprising a cell wall and comprising a protein content between 10% and 90% by weight of the fraction.

### The Method

According to some embodiments, there is provided a method for separating a protein from a microorganism comprising a cell wall. In some embodiments, the method comprises applying pressure between 150 bars and 1500 bars to the microorganism and receiving a first fraction comprising soluble proteins and a second fraction comprising insoluble proteins.

According to some embodiments, there is provided a method for optimizing a protein content extracted from a microorganism comprising a cell wall, the method comprising: a) breaking the cell wall of the microorganism to a particle size suitable for enzymatic reaction; and b) receiving a first fraction comprising soluble proteins and a second fraction comprising insoluble proteins, wherein any one of the first and the second fraction comprises a protein content between 10% and 90% by weight of the fraction, thereby optimizing the protein content extracted from the microorganism comprising a cell wall.

In some embodiments, the method comprises applying a pressure between 100 bars and 1500 bars, 150 bars and 1500 bars, 250 bars and 1500 bars, 450 bars and 1500 bars, 490 bars and 1500 bars, 500 bars and 1500 bars, 550 bars and 1500 bars, 700 bars and 1500 bars, 1000 bars and 1500 bars, 100 bars and 1200 bars, 150 bars and 1200 bars, 250 bars and 1200 bars, 450 bars and 1200 bars, 490 bars and 1200 bars, 500 bars and 1200 bars, 550 bars and 1200 bars, 700 bars and 1200 bars, 1000 bars and 1200 bars, 50 bars and 900 bars, 100 bars and 900 bars, 250 bars and 900 bars, 450 bars and 900 bars, 490 bars and 900 bars, 500 bars and 900 bars, 550 bars and 900 bars, 700 bars and 900 bars, 450 bars and 700 bars, 490 bars and 700 bars, 500 bars and 700 bars, or between 550 bars and 700 bars, to the microorganism, including any range therebetween. Each possibility represents a separate embodiment of the invention.

In some embodiments, any one of the first and the second fraction comprises a protein content between 10% and 90% by weight of the fraction.

In some embodiments, any one of the first and the second fraction comprises a protein content between 10% and 90%, 12% and 90%, 15% and 90%, 20% and 90%, 50% and 90%, 50% and 90%, 10% and 85%, 12% and 85%, 15% and 85%, 20% and 85%, 50% and 85%, 50% and 85%, 10% and 80%, 12% and 80%, 15% and 80%, 20% and 80%, 50% and 80%, 50% and 80%, 10% and 75%, 12% and 75%, 15% and 75%, 20% and 75%, 50% and 75%, or between 50% and 75%, by weight of the fraction, including any range therebetween. Each possibility represents a separate embodiment of the invention.

In some embodiments, the particle size suitable for enzymatic reaction is between 2 μm and 10 μm, 3 μm and 10 μm, 4 μm and 10 μm, 5 μm and 10 μm, 6 μm and 10 μm, 2 μm and 7 μm, 3 μm and 7 μm, 4 μm and 7 μm, or 2 μm and 5 μm, including any range therebetween. Each possibility represents a separate embodiment of the invention.

In some embodiments, breaking the cell wall comprises applying any one of a) pressure between 150 bars and 1500 bars; b) temperature between 50° C. and 100° C.; c) ultrasonication, d) bead mill homogenization, or any combination thereof to the microorganism.

According to some embodiments, there is provided a method for separating a protein from a microorganism comprising a cell wall, comprising applying pressure between 150 bars and 1500 bars to the microorganism and receiving a first fraction comprising soluble proteins and a second fraction comprising insoluble proteins, wherein any one of the first and the second fraction comprises a protein content between 10% and 90% by weight of the fraction, thereby separating a protein from the microorganism comprising a cell wall.

In some embodiments, the first fraction, the second fraction, or both is suitable for extrusion.

As used herein the term “extrusion” refers to a high temperature, high pressure, short time process that transforms a variety of raw materials and ingredients into modified intermediate and finish products.

In some embodiments, the microorganism is a bacteria, a fungi or a microalgae.

Non-limiting examples of suitable bacteria according to the present invention include Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, Weissella, Escherichia coli, Acetobacter, Clostridium thermocellum, Anoxybacillus flavithermus, Geobacillus, Micrococcus luteus, or any combination thereof.

As used herein, the term “microalgae” means any unicellular, photosynthetic microorganism, whether wild type or genetically modified microalgae.

As used herein “fungi” refers to any member of the group of eukaryotic organisms that include microorganisms such as yeasts, molds, and mushrooms.

In some embodiments, the fungi comprises mycelium or is a mycelium producing microorganism. Non-limiting examples include Fusarium venenatum, Aspergillus oryzae, Monascus purpureus, Neurospora intermedia, and Zygomycota (Rhizopus oryzae).

In some embodiments, the fungi is a yeast.

As used herein the term “yeast” refers to eukaryotic, single-celled microorganisms from the phyla Ascomycota and Basidiomycota. An exemplary yeast is budding yeast from the order Saccharomycetales. A particular example of yeast is Saccharomyces spp., including but not limited to Saccharomyces cerevisiae. In some embodiments, the yeast comprises Saccharomyces cerevisiae, Streptomyces natalensis, Streptomyces chattanoogensis, Saccharomyces fragilis, Candida utilis, Candida guilliermondii, Candida lipolityca, Cyberlindnera jadinii, Pichia pastoris, or any combination thereof.

In some embodiments, the yeast is derived from downstream food-related industries. In some embodiments, the yeast material is derived from beer downstream. In some embodiments, the yeast material is derived from yeast extract downstream. In some embodiments, the yeast comprises spent yeast, instant yeast, inactive yeast, live yeast, or any combination thereof.

In some embodiments, any one of the first fraction and the second fraction comprises a particle characterized by an average particle size between 1 μm and 100 μm, 1 μm and 95 μm, 1 μm and 90 μm, 1 μm and 89 μm, 1 μm and 80 μm, 1 μm and 50 μm, 2 μm and 50 μm, 3 μm and 50 μm, 4 μm and 50 μm, 5 μm and 50 μm, 6 μm and 50 μm, 1 μm and 40 μm, 2 μm and 40 μm, 3 μm and 40 μm, 4 μm and 40 μm, 5 μm and 40 μm, 6 μm and 40 μm, 1 μm and 10 μm, 2 μm and 10 μm, 3 μm and 10 μm, 4 μm and 10 μm, 5 μm and 10 μm, 6 μm and 10 μm, 1 μm and 9 μm, 2 μm and 9 μm, 3 μm and 9 μm, 4 μm and 9 μm, 5 μm and 9 μm, 6 μm and 9 μm, 1 μm and 8 μm, 2 μm and 8 μm, 3 μm and 8 μm, 4 μm and 8 μm, 5 μm and 8 μm, 6 μm and 8 μm, 1 μm and 6 μm, 2 μm and 6 μm, 3 μm and 6 μm, or between 4 μm and 6 μm, including any range therebetween. Each possibility represents a separate embodiment of the invention.

In some embodiments, any one of the first fraction and the second fraction comprises a particle characterized by a maximum particle size between 1 μm and 100 μm, 1 μm and 95 μm, 1 μm and 90 μm, 1 μm and 89 μm, 1 μm and 80 μm, 1 μm and 50 μm, 2 μm and 50 μm, 3 μm and 50 μm, 4 μm and 50 μm, 5 μm and 50 μm, 6 μm and 50 μm, 1 μm and 40 μm, 2 μm and 40 μm, 3 μm and 40 μm, 4 μm and 40 μm, 5 μm and 40 μm, 6 μm and 40 μm, 1 μm and 10 μm, 2 μm and 10 μm, 3 μm and 10 μm, 4 μm and 10 μm, 5 μm and 10 μm, 6 μm and 10 μm, 1 μm and 9 μm, 2 μm and 9 μm, 3 μm and 9 μm, 4 μm and 9 μm, 5 μm and 9 μm, 6 μm and 9 μm, 1 μm and 8 μm, 2 μm and 8 μm, 3 μm and 8 μm, 4 μm and 8 μm, 5 μm and 8 μm, 6 μm and 8 μm, 1 μm and 6 μm, 2 μm and 6 μm, 3 μm and 6 μm, or between 4 μm and 6 μm, including any range therebetween. Each possibility represents a separate embodiment of the invention.

In some embodiments, the protein is characterized by a molecular weight between 1 kDa and 250 kDa, between 5 kDa and 250 kDa, between 10 kDa and 250 kDa, between 15 kDa and 250 kDa, between 20 kDa and 250 kDa, between 25 kDa and 250 kDa, between 30 kDa and 250 kDa, between 1 kDa and 200 kDa, between 5 kDa and 200 kDa, between 10 kDa and 200 kDa, between 15 kDa and 200 kDa, between 20 kDa and 200 kDa, between 25 kDa and 200 kDa, or between 30 kDa and 200 kDa, including any range therebetween. Each possibility represents a separate embodiment of the invention.

In some embodiments, the insoluble protein is characterized by an aqueous solubility of less than 300 g/L, less than 280 g/L, less than 250 g/L, less than 220 g/L, less than 200 g/L, less than 150 g/L, or less than 100 g/L, including any value therebetween. Each possibility represents a separate embodiment of the invention.

According to some embodiments, there is provided a method for obtaining a yeast material from yeast comprising a protein content between 10% and 90% by weight of the yeast material. In some embodiments, the method comprises the steps: a. providing a yeast; and b. applying a pressure between 150 bars and 1500 bars to the yeast, thereby obtaining the yeast material from yeast.

The term “yeast material” as used herein, refers to any compound constituting, secreted, derived or produced by yeast. In some embodiments, yeast material is a carbon-based material.

In some embodiments, the yeast material comprises: a whole yeast, a yeast extract, a yeast biomass, a yeast homogenate, a yeast filtrate, a yeast concentrate, any fraction thereof, or any combination thereof. In some embodiments, the yeast material is selected from the group consisting of: a whole yeast, a yeast biomass, a yeast filtrate, a yeast concentrate, any fraction thereof, and any combination thereof. In some embodiments, the yeast material comprises or consists of a whole yeast. In some embodiments, the yeast material is devoid of a yeast extract.

In some embodiments, the yeast material comprises a powder, solution, suspension, or any combination thereof.

In some embodiments, step b comprises applying a pressure between 100 bars and 1500 bars, 150 bars and 1500 bars, 250 bars and 1500 bars, 450 bars and 1500 bars, 490 bars and 1500 bars, 500 bars and 1500 bars, 550 bars and 1500 bars, 700 bars and 1500 bars, 1000 bars and 1500 bars, 100 bars and 1200 bars, 150 bars and 1200 bars, 250 bars and 1200 bars, 450 bars and 1200 bars, 490 bars and 1200 bars, 500 bars and 1200 bars, 550 bars and 1200 bars, 700 bars and 1200 bars, 1000 bars and 1200 bars, 100 bars and 900 bars, 150 bars and 900 bars, 250 bars and 900 bars, 450 bars and 900 bars, 490 bars and 900 bars, 500 bars and 900 bars, 550 bars and 900 bars, 700 bars and 900 bars, 450 bars and 700 bars, 490 bars and 700 bars, 500 bars and 700 bars, or between 550 bars and 700 bars, to the yeast, including any range therebetween. Each possibility represents a separate embodiment of the invention.

In some embodiments, the yeast material comprises a protein content between 10% and 90%, 12% and 90%, 15% and 90%, 20% and 90%, 50% and 90%, 50% and 90%, 10% and 85%, 12% and 85%, 15% and 85%, 20% and 85%, 50% and 85%, 50% and 85%, 10% and 80%, 12% and 80%, 15% and 80%, 20% and 80%, 50% and 80%, 50% and 80%, 10% and 75%, 12% and 75%, 15% and 75%, 20% and 75%, 50% and 75%, or between 50% and 75%, by weight of the yeast material, including any range therebetween. Each possibility represents a separate embodiment of the invention.

In some embodiments, the yeast material comprises a nitrogen content between 1% and 15%, 4% and 15%, 5% and 15%, 6% and 15%, 7% and 15%, 1% and 10%, 4% and 10%, 5% and 10%, 6% and 10%, 7% and 10%, 4% and 7%, or between 5% and 7%, by weight of the yeast material, including any range therebetween. Each possibility represents a separate embodiment of the invention.

In some embodiments, the yeast material was obtained from a solid fraction by applying a pressure between 150 bar and 1500 bar. In some embodiments, a yeast material obtained from a solid fraction by applying a pressure between 150 bar and 1500 bar, comprises a protein content between 40% and 70% by weight of the yeast material. In some embodiments, the protein content is calculated by conversion of the nitrogen content. In some embodiments, the protein content is calculated by using 6.25 nitrogen-protein conversion coefficient.

In some embodiments, the yeast material was obtained from a liquid fraction by applying a pressure between 250 bar and 700 bar. In some embodiments, a yeast material obtained from a liquid fraction by applying a pressure between 250 bar and 700 bar, comprises a protein content between 15% and 50% by weight of the yeast material. In some embodiments, the protein content is calculated by conversion of the nitrogen content. In some embodiments, the protein content is calculated by using 6.25 nitrogen-protein conversion coefficient.

In some embodiments, the yeast material is suitable for extrusion.

In some embodiments, the yeast material is characterized by an average particle size between 1 μm and 100 μm, 1 μm and 95 μm, 1 μm and 90 μm, 1 μm and 89 μm, 1 μm and 80 μm, 1 μm and 50 μm, 2 μm and 50 μm, 3 μm and 50 μm, 4 μm and 50 μm, 5 μm and 50 μm, 6 μm and 50 μm, 1 μm and 40 μm, 2 μm and 40 μm, 3 μm and 40 μm, 4 μm and 40 μm, 5 μm and 40 μm, 6 μm and 40 μm, 1 μm and 10 μm, 2 μm and 10 μm, 3 μm and 10 μm, 4 μm and 10 μm, 5 μm and 10 μm, 6 μm and 10 μm, 1 μm and 9 μm, 2 μm and 9 μm, 3 μm and 9 μm, 4 μm and 9 μm, 5 μm and 9 μm, 6 μm and 9 μm, 1 μm and 8 μm, 2 μm and 8 μm, 3 μm and 8 μm, 4 μm and 8 μm, 5 μm and 8 μm, 6 μm and 8 μm, 1 μm and 6 μm, 2 μm and 6 μm, 3 μm and 6 μm, or between 4 μm and 6 μm, including any range therebetween. Each possibility represents a separate embodiment of the invention.

In some embodiments, applying a pressure comprises high-pressure homogenization.

As used herein, the term “high-pressure homogenization” refers to a process in which a sample (primarily liquid sample) is forced though a system which subjects it to any one of a number of forces which is intended to homogenize the sample and/or reduce the particle sizes of any components within it. Depending on the setup of a particular system, high-pressure homogenization can be performed and operate on any combination of shear forces, impact, and cavitation. In some embodiments, the process may be carried out through a valve (homogenizing valve) or by milling cells, with application of pressure.

The term “milling” as used herein, refers to methods by which an external force is applied to a solid that leads to its break-up into smaller particles. In one embodiment milling refers to wet grinding carried out using methods as a roller ointment mill, tumbling ball mill, vibratory ball mill, a planetary ball mill, a centrifugal fluid mill, an agitating beads mill, a flow conduit beads mill, an annular gap beads mill, and wet jet mill. In one embodiment milling refers to dry grinding by compression or by friction, using methods as a jet mill, a hammer mill, a shearing mill, a roller mill, a shock shearing mill, a ball mill, and a tumbling mill. In one embodiment milling refers to wet processes for preventing the condensation of the particles so formed, and obtaining highly dispersed particles.

In some embodiments, the method further comprises a step comprising removing a fiber from the cell wall or portion thereof.

In some embodiments, the step of removing a fiber from the cell wall or portion thereof is performed before applying a pressure, during applying a pressure, or after applying a pressure, wherein applying a pressure is as described hereinabove.

In some embodiments, removing a fiber comprises chemical hydrolysis, enzymatic hydrolysis, or both.

In some embodiments, the method further comprises a hydrolysis step. In some embodiments, the method further comprises an enzymatic hydrolysis step.

In some embodiments, the enzymatic hydrolysis comprises contacting the microorganism with an enzyme, wherein the enzyme is selected from the group consisting of: lipase, phospolipase, polygalacturonase, chitinase, pectinase, β-glucanase, protease, arabinanase, pectinase, endo-amalyase, endo-β-glycanase, hydrolase, cellulose, arabanase, cellulase, hemicellulase, xylanase, laccase, mannanase, exo-endo-protease, or any combination thereof.

In some embodiments, the method further comprises a step comprising contacting the yeast material with an enzyme. In some embodiments, the enzyme is selected from lipase, phospolipase, polygalacturonase, chitinase, pectinase, β-glucanase, protease, arabinanase, pectinase, endo-amalyase, endo-β-glycanase, hydrolase, cellulose, arabanase, cellulase, hemicellulase, xylanase, laccase, mannanase, exo-endo-protease, or any combination thereof.

In some embodiments, the enzyme comprises phospolipase, polygalacturonase, chitinase, pectinase, β-glucanase, protease and arabinanase. In some embodiments, the enzyme comprises i. phospolipase, and ii. polygalacturonase, chitinase, pectinase, β-glucanase, protease and arabinanase. In some embodiments, i. phospolipase, and ii. polygalacturonase, chitinase, pectinase, β-glucanase, protease and arabinanase are used at a weight per weight ratio of 1:1.

In some embodiments, the enzymatic hydrolysis is performed at a pH between 3.5 and 7, 4 and 7, 4.5 and 7, 3.5 and 6.5, 4 and 6.5, or 4.5 and 6.5, including any range therebetween. Each possibility represents a separate embodiment of the invention.

In some embodiments, the method comprises repeating applying pressure between 150 bars and 1500 bars. In some embodiments, the method comprises repeating applying pressure between 150 bars and 1500 bars, between 1 and 5 times, 1 and 4 times, 1 and 3 times, 1 and 2 times, 2 and 5 times, 2 and 4 times, 2 and 3 times, 3 and 5 times, or between 3 and 4 times, including any range therebetween. Each possibility represents a separate embodiment of the invention.

In some embodiments, a first fraction and a second fraction are separated. In some embodiments, a first fraction and a second fraction are separated by phase separation. Phase separation can be performed by any means known to one skilled in the art. In some embodiments, phase separation is performed by centrifugation.

In some embodiments, the method further comprises an enzyme deactivation step. Enzyme deactivation is known to those skilled in the art. In some embodiments, enzyme deactivation is performed at a temperature between 50° C. and 100° C., 60° C. and 100° C., 65° C. and 100° C., 70° C. and 100° C., 75° C. and 100° C., 80° C. and 100° C., 85° C. and 100° C., or between 90° C. and 100° C., including any range therebetween. Each possibility represents a separate embodiment of the invention.

In some embodiments, the method further comprises a step comprising cleaning, grinding, milling, washing, drying or any combination thereof, the microorganism. In some embodiments, the method further comprises an initial step comprising cleaning, grinding, milling, washing, drying or any combination thereof, the microorganism.

In some embodiments, any one of the first fraction and the second fraction is in the form of a powder, solution, suspension, or any combination thereof.

In some embodiments, the method further comprises a step of extracting the microorganism between 1 and 5 times, 1 and 4 times, 1 and 3 times, 1 and 2 times, 2 and 5 times, 2 and 4 times, 2 and 3 times, 3 and 5 times, or between 3 and 4 times, including any range therebetween. Each possibility represents a separate embodiment of the invention.

In some embodiments, the method further comprises a step comprising isolating, purifying, concentrating, or any combination thereof, a protein from the first fraction, the second fraction or both.

In some embodiments, the method is devoid of solvents.

### The Composition

According to some embodiments, there is provided a composition comprising a first fraction, a second fraction or both, derived from a microorganism comprising a cell wall and comprising a protein content between 10% and 90% by weight of the fraction, obtained by the method described hereinabove.

According to some embodiments, there is provided a composition comprising a yeast material comprising a protein content between 10% and 90% by weight of the yeast material, obtained by the method described hereinabove.

In some embodiments, the composition was obtained from a solid fraction by applying a pressure between 250 bar and 1500 bar. In some embodiments, a composition obtained from a solid fraction by applying a pressure between 250 bar and 1500 bar, comprises a protein content between 30% and 70% by weight of the yeast material. In some embodiments, the protein content is calculated by conversion of the nitrogen content. In some embodiments, the protein content is calculated by using 6.25 nitrogen-protein conversion coefficient.

In some embodiments, the composition comprises a yeast material as described herein comprising a nitrogen content between 1% and 15%, 4% and 15%, 5% and 15%, 6% and 15%, 7% and 15%, 1% and 10%, 4% and 10%, 5% and 10%, 6% and 10%, 7% and 10%, 4% and 7%, or between 5% and 7%, by weight of the yeast material, including any range therebetween. Each possibility represents a separate embodiment of the invention.

According to some embodiments, there is provided a composition comprising more than 50% by dry weight of a protein derived from a microorganism comprising a cell wall. In some embodiments, the composition comprises more than 50%, more than 51%, more than 55%, more than 60%, or more than 62%, by dry weight of a protein derived from a microorganism comprising a cell wall.

In some embodiments, the protein is characterized by a molecular weight between 1 kDa and 250 kDa, between 5 kDa and 250 kDa, between 10 kDa and 250 kDa, between 15 kDa and 250 kDa, between 20 kDa and 250 kDa, between 25 kDa and 250 kDa, between 30 kDa and 250 kDa, between 1 kDa and 200 kDa, between 5 kDa and 200 kDa, between 10 kDa and 200 kDa, between 15 kDa and 200 kDa, between 20 kDa and 200 kDa, between 25 kDa and 200 kDa, or between 30 kDa and 200 kDa, including any range therebetween. Each possibility represents a separate embodiment of the invention.

In some embodiments, the composition is characterized by an aqueous solubility of less than 300 g/L, less than 280 g/L, less than 250 g/L, less than 220 g/L, less than 200 g/L, less than 150 g/L, or less than 100 g/L, including any value therebetween. Each possibility represents a separate embodiment of the invention.

In some embodiments, the protein is characterized by an amino acid profile presented in Table 13.

In some embodiments, the composition is in the form of a powder characterized by an average particle size between 1 μm and 100 μm, 1 μm and 95 μm, 1 μm and 90 μm, 1 μm and 89 μm, 1 μm and 80 μm, 1 μm and 50 μm, between 2 μm and 50 μm, between 4 μm and 50 μm, between 5 μm and 50 μm, between 10 μm and 50 μm, between 15 μm and 50 μm, between 20 μm and 50 μm, between 1 μm and 45 μm, between 2 μm and 45 μm, between 4 μm and 45 μm, between 5 μm and 45 μm, between 10 μm and 45 μm, between 15 μm and 45 μm, between 20 μm and 45 μm, between 1 μm and 40 μm, between 2 μm and 40 μm, between 4 μm and 40 μm, between 5 μm and 40 μm, between 10 μm and 40 μm, between 15 μm and 40 μm, or between 20 μm and 40 μm, including any range therebetween. Each possibility represents a separate embodiment of the invention.

In some embodiments, the composition comprises a fiber content between 5% and 20%, between 6% and 20%, between 7% and 20%, between 5% and 15%, between 6% and 15%, or between 7% and 15% by dry weight, including any range therebetween. Each possibility represents a separate embodiment of the invention.

In some embodiments, the composition comprises an ash content of less than 5%, less than 4.5%, less than 4%, less than 3.9%, or less than 3%, including any value therebetween. Each possibility represents a separate embodiment of the invention.

In some embodiments, the composition is characterized by a protein digestibility-corrected amino acid score (PDCAAS) between 0.7 and 1, between 0.75 and 1, between 0.8 and 1, between 0.85 and 1, or between 0.9 and 1, including any value therebetween. Each possibility represents a separate embodiment of the invention.

In some embodiments, the microorganism is a bacteria, a fungi or a microalgae, as described hereinabove. In some embodiments, the fungi is a yeast.

In some embodiments, the yeast comprises Saccharomyces cerevisiae, Streptomyces natalensis, Streptomyces chattanoogensis, Saccharomyces fragilis, Candida utilis, Candida guilliermondii, Candida lipolityca, Cyberlindnera jadinii, Pichia pastoris, or any combination thereof.

In some embodiments, the yeast is derived from downstream food-related industries. In some embodiments, the yeast comprises spent yeast, instant yeast, inactive yeast, live yeast, or any combination thereof.

According to some embodiments, there is provided a food product comprising the composition described hereinabove.

As used herein, the term “food product” refers to a material, a substance, or an additive, which can be used as food, or which can be added to food. Typically, the food product is any composition that an animal, preferably a mammal such as a human, may consume as part of its diet. In some embodiments, food product refers to a food supplement.

In some embodiments, the food product is characterized as being suitable for use as an equivalent product to meat, eggs, cheese, dairy products, meat substitute products, plant-based products, or any combination thereof.

In some embodiments, the food product is characterized by at least 60% of the consistency of an equivalent product prepared using eggs. In some embodiments, the food product is characterized by at least 60%, at least 65%, at least 70%, at least 75%, at least 80%, at least 85%, at least 90%, or at least 95%, of the consistency of an equivalent product prepared using meat, eggs, dairy products, or any combination thereof, including any value therebetween. Each possibility represents a separate embodiment of the invention.

In some embodiments, the food product comprises meat, eggs, dairy products, or any combination thereof.

In some embodiments, the food product is devoid of meat, eggs, dairy products, or any combination thereof. In one embodiment, “free of” is “devoid of” or essentially “devoid of”.

As used herein, the terms “egg” “meat”, “dairy products”, for example, as used when describing a “product devoid of meat, eggs, dairy products,” refers to an animal product or any component of an animal product.

In some embodiments, the food product is a vegetarian food product. In some embodiments, the food product is a vegan food product.

As used herein, the term “vegan” refers to properties of the components, and indicates that the components are not sourced from or derived from an animal or animal product. As such, the components that are “vegan” are free of any animal products or animal byproducts. What constitutes an animal product or byproduct is well known in this field, and to those following a vegetarian or vegan diet. In particular, the term “animal product” refers to any animal parts, animal byproducts, or products produced by an animal. Some examples of materials that would be considered “animal products” include those parts of the animal that are consumable or typically prepared for consumption by humans (including, e.g., fat, flesh, blood, etc.). Products produced by an animal are also considered “animal products” as used herein, and refer to the products produced by an animal without slaughtering the animal, (e.g., milk, eggs, honey, etc.). “Animal byproducts” are products that are typically not consumable by themselves but are the byproducts of slaughtering animals for consumption, e.g., bones, carcasses, etc. However, animal byproducts are often processed into human consumable foodstuffs, some well-known examples of which include gelatin, casein, whey, rennet, etc. As used herein, these processed animal byproducts (e.g., gelatin, casein, whey, rennet, etc.) are encompassed by the term “animal byproducts.” As described herein, “vegan” and “plant-based” components or ingredients are substantially free (or in some embodiments, completely free) of such animal products and byproducts.

In some embodiments, compositions and food products as described herein are suitable for a vegan diet and/or a vegetarian diet. For example, in embodiments in which the composition is suitable for a vegan diet, the composition may include primarily plant-based components such that the composition contains substantially no animal products, animal byproducts, or substantially no components derived from these animal sources.

In some embodiments, the food product is a ready to use product. In some embodiments, the food product is a frozen food product. In some embodiments, the food product is suitable for cooking via e.g. heating, frying, and backing.

In some embodiments, a food product or composition as described herein, comprises one or more flavoring agents. In some embodiments, a food product or composition as described herein, comprises yeast, sugar, salt, and any combination thereof. Various natural or artificial flavoring agents are known to those skilled in the art, and can include, for example, salt, spices, sugar, sweeteners, monosodium glutamate, sulfuric flavoring agents such as black salt, or other flavoring agents.

In some embodiments, a food product or composition as described herein, comprises one or more coloring agents. Various natural or artificial coloring agents are known to those skilled in the art, and can include, for example, carotenoids such as beta-carotene, turmeric, annatto, mango yellow, or palm-based oils.

In some embodiments, a food product or composition as described herein, further comprises an emulsifier, a thickener, an oil, or any combination thereof.

In some embodiments, the oil is a vegetable-based oil. Examples of vegetable oils that may be used according to the present invention include, but are not limited to, soybean oil, safflower oil, linseed oil, corn oil, sunflower oil, olive oil, canola oil, sesame oil, cottonseed oil, palm oil, rapeseed oil, tung oil, or a blend of any of these oils. Alternatively, any partially hydrogenated vegetable oils or genetically modified vegetable oils can be used. Examples of partially hydrogenated vegetable oils or genetically modified vegetable oils include, but are not limited to, high oleic safflower oil, high oleic soybean oil, high oleic peanut oil, high oleic sunflower oil and high erucic rapeseed oil (crambe oil).

### General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Process

Yeast material was mixed with water at 25° C. for 30 min. Mechanical disruption was used to break the cell walls according to the conditions described. Optionally, enzyme treatment was performed followed by centrifugation. The pellet was separated from the supernatant and dried, affording the final protein powder.

### Example 1

### Yeast Cell Wall Breaking Via Dry and Wet Fraction Techniques

### Materials and Methods

### Yeast

The yeasts used for the experiments were SAF Instant Red provided by Lesaffre. It is an instant dry yeast, universally suitable for all dough and guides, for direct addition to the dough. Table 1 summarizes information from the technical data sheet.

TABLE 1
Yeast data sheet information

| Product | Value |
|---|---|
| Fat (g) | 5.7 |
| of which saturated fatty acids (g) | 0.9 |
| Carbohydrates | 19.0 |
| of which sugar (g) | 14.0 |
| Dietary fibers (g) | 27.0 |
| Protein (g) | 43.7 |
| Salt (g) | 0.3 |

### Enzymes

Table 2 describes the enzymes used for the experiments.

TABLE 2
Properties of enzymes used in the present experiments

| Provider | Activity | Enzyme |
|---|---|---|
| Novozyme | Endo-β-glucanase | Viscozyme L |
| | hydrolyzing | |
| | (1,3)- or (1,4)-linkages | |
| | in -D-glucans | |
| Novozyme | (1,4)-α-D- | Pectinex/ |
| | galactosiduronic | Pectinase |
| | linkages in pectate and | Ultra SP-L |
| | other galacturonans | |
| Novozyme | Serine endo-peptidase | Alcalase 2.4 |
| | (mainly subtilisin A) | L FG |

### Analysis

### Total Nitrogen

Total nitrogen was determined by Kjeldahl (Tecator™ with Kjeltec 8400, Foss® system) according to internal procedure PR-16027 (NF EN ISO 5983-2). Protein content was esteemed by using 6.25 as nitrogen-protein conversion coefficient.

### Dry Matter

Dry matter content was determined by following internal procedure PR-14032 by using automatic PrepAsh (serie 340, modele 219, Precisa®) system. Dry matter content was determined after drying at 105° C., up to have constant weight.

### Particle Size Distribution

Particle size distribution (PSD) was determined by following internal procedure PR-17014 by using Mastersizer 3000 (Malvern) using the Hydro MV modulus.

### Electrophoresis

Analyses were realized by internal procedure P14027 using SDS and β-mercaptoethanol.

### Sample Preparing

To realize nitrogen and dry matter analyses, the samples were previously centrifuged at 4 000×g for 10 minutes in lab centrifuge (Jouan). To realize particle size distribution, full sample was used.

### Particle Size Distribution Results

Processes to lyse yeast cells were implemented to extract cytoplasmic proteins by high-pressure homogenizer and enzymatic hydrolysis. Each experiment was evaluated by granulometry pointing out an effect of the applied process and Table 3 shows d10, d50 d91 extrapolated values of particle size distributions (PSD).

TABLE 3
Particle size distributions (PSD)

| Sample Name | Conditions | d10 (μm) | d50 (μm) | d90 (μm) |
|---|---|---|---|---|
| Initial suspension | | 3.09 | 12.6 | 38.5 |
| C5665-11 | HP H 500 bar, 1 passe | 2.79 | 4.36 | 6.61 |
| C5665-12 | HP H 500 bar, 2 passe | 2.78 | 4.36 | 6.61 |
| C5665-13 | HP H 500 bar, 3 passe | 2.76 | 4.35 | 6.62 |
| C5665-21 | HP H 1000 bar, 1 passe | 2.59 | 4.19 | 6.46 |
| C5665-51 | HP H 250 bar, 1 passe | 2.88 | 4.50 | 6.83 |
| C5665-31 | Hydrolyse, Viscozyme L | 3.29 | 6.04 | 13.5 |
| C5665-32 | Hydrolyse + HP H 500 bar, 1 passe | 3.40 | 4.92 | 6.95 |
| C5665-41 | Hydrolysis, enzymatic cocktail | 2.28 | 3.92 | 6.24 |
| C5665-42 | Hydrolysis, HP H 250 bar, 1 passe | 2.10 | 3.81 | 5.99 |
| C5665-43 | Hydrolysis, HP H 500 bar, 1 passe | 2.09 | 3.79 | 5.97 |

d10: diameter at which 10% of a sample's mass is comprised of smaller particles (ie 10% of the particles have a diameter smaller than or equal to d10 value); d50: diameter at which 50% of a sample's mass is comprised of smaller particles (ie 50% of the particles have a diameter smaller than or equal to d50 value); d90: diameter at which 90% of a sample's mass is comprised of smaller particles (ie 90% of the particles have a diameter smaller than or equal to d90 value).

The data shows similar values for the “d” indexes for the different experiments performed by using the homogenizer. Exceptions are shown by hydrolysis process, where higher values of d50 and d90 indexes are shown in the case of viscozyme hydrolysis (sample C5665-31), whereas enzymatic cocktails show lower values of d10 and d50. Overall, PSD does not show important differences among treatments.

### Protein Content

Protein content (N×6.25) (/DM) highlights differences among treatments and FIGS. 3A-B summarize the progressions.

The number of passes (from 1 to 3) at constant pressure leads to higher protein purity in the supernatant. In the case of the experiment realized at 500 bars, the purity increase was of 47%. Increase of the pressure (from 250 to 500 and to 1 000 bars) at constant number of passes can increase protein purity in the supernatant. Experiments using 1 passe realized at 500 bars show an increase of purity of 7% in the supernatant, compared with experiment realized at 250 bars. Experiments with 3 passes realized at 1 000 bars increases protein purity of 29% in the supernatant when comparing to application of 3 passes at 500 bars (FIG. 3A).

Enzymatic hydrolysis realized for 3 hours by β-glucanase (viscozyme L) afforded 36% of protein purity (FIG. 3B). Coupling this enzymatic treatment with homogenization (500 bars, 1 passe) lead to a purity increase up to 50% (+36%). Enzymatic hydrolysis realized for 3 hours by enzymatic cocktails (viscozyme L, pectinase and alcalase) produced a supernatant having 28% of protein purity (8% less of what observed by viscozyme alone). The application of 250 bars did not point out any improvement. Application of 500 bars increased protein purity of 31% in the supernatant, corresponding to +10%.

### Protein Yield

Comparison of protein yields from all the experiments are summarized in Table 4.

TABLE 4
Summary of protein yields from different experimental conditions.

| | | | Proteins | | | | |
|---|---|---|---|---|---|---|---|
| | | Type of | (N × 6.25) | DM | Ash/DM | Proteins/DM | Yield |
| Experiment | Conditions | sample | (%) | (%) | (%) | (%) | (%) |
| Initial | | Initial | 4.7% | 10.9% | 5% | 43% | |
| Suspension | | suspension | | | | | |
| HPH | 1 passe | Supernatant | 0.7% | 3.9% | 9% | 18% | 10% |
| 250 bars | | Pellet | 18.2% | 34.1% | 4% | 53% | |
| HPH | 1 passe | Supernatant | 0.7% | 3.7% | 11% | 19% | |
| 500 bars | | Pellet | 17.4% | 31.6% | 4% | 55% | |
| | 3 passes | Supernatant | 0.9% | 3.3% | 13% | 28% | 13% |
| | | Pellet | 17.3% | 31.1% | 4% | 56% | |
| HPH | 3 passes | Supernatant | 2.1% | 5.7% | 9% | 36% | 28% |
| 1000 bars | | Pellet | 12.9% | 25.4% | 4% | 51% | |
| Enzyme | 3 hours | Supernatant | 0.6% | 1.7% | 19% | 36% | |
| hydrolysis + | Viscozyme | Pellet | 13.4% | 28.0% | 5% | 48% | |
| HPH | L | | | | | | |
| 500 bars | 1 passe | Supernatant | 1.2% | 2.5% | 14% | 49% | 15% |
| | | Pellet | 11.2% | 26.9% | 5% | 42% | |
| Enzyme | 3 hours | Supernatant | 1.3% | 4.5% | 13% | 28% | |
| hydrolysis + | Viscozyme, | Pellet | 15.7% | 34.8% | 3% | 45% | |
| HPH | Pectinase, | | | | | | |
| 250 bars | Alcalase | | | | | | |
| | 1 passe | Supernatant | 1.1% | 4.0% | 14% | 28% | 14% |
| | | Pellet | 18.5% | 36.1% | 3% | 51% | |
| Enzyme | 3 hours | Supernatant | 1.3% | 4.5% | 13% | 28% | |
| hydrolysis + | Viscozyme, | Pellet | 15.7% | 34.8% | 3% | 45% | |
| HPH | Pectinase, | | | | | | |
| 500 bars | Alcalase | | | | | | |
| | 1 passe | Supernatant | 1.3% | 4.3% | 14% | 31% | 20% |
| | | Pellet | 18.5% | 35.7% | 3% | 52% | |

In terms of yield, best tested condition were 1 000 bars, 3 passes, as yield increases of 115% compared to 500 bars −3 passes experiments. However, protein purity has to be considered and combination of hydrolysis by viscozyme alone and homogenization shows highest observed purity (49%). Experiments combining hydrolysis and homogenization point out that Viscozyme alone can release higher amount of proteins compared with experiment realized by enzymatic mix (+58%).

### Example 2

### Method Optimization

Different mechanical disruption methods were successfully used in several yeast raw materials, including heat, bead mill, ultrasound and high pressure homogenizer.

The use of the homogenizer proved to be a very efficient method (at pressure between 200-750 bars) to mechanical disrupt the cells on one hand and to fractionate as many proteins as possible into the insoluble phase on the other hand.

Table 5 presents the protein content obtained from the same yeast stream using different mechanical disruption methods.

TABLE 5
Protein content after mechanical disruption

| Mechanical | | Bead | ultra- | High pressure |
|---|---|---|---|---|
| disruption step | heat | mill | sound | homogenizer (500 bar) |
| Protein in the final | 60-70 | 60-70 | 60-70 | 65-75 |
| product (%) | | | | |

### Enzymatic Step

Different enzymes were successfully used alone and in combinations. The enzymes include: a) Lecitase—a Lipase (phospolypase); b) Ban 48L0—endo amalyase with beta glucanase side activity; c) Pectinex Ultra SPL—polygalacturonase; d) Vino Taste Pro—polygalacturonase with chitinase side activity (pectinases, beta-glucanase, protease and an arabinanase); e) Viscozyme—endo beta glycanase (complex of enzymes); f) Alcalase—protein hydrolase; and g) Flavorzyme-protein hydrolase. A summary of results is presented in Table 6.

TABLE 6
Protein content after enzymatic step

| | 5 | 4 | 4 | 4 | 4 | 4 | 2 |
|---|---|---|---|---|---|---|---|
| Enzymatic | enzymes | enzymes | enzymes | enzymes | enzymes | enzymes | enzymes |
| combo | (a − e) | (b − e) | (a − d) | (a, c − e) | (a − b, d − e) | (a − c, e) | (a + d) |
| Protein in the | 65-75 | 55-65 | 65-75 | 65-75 | 65-75 | 55-65 | 65-75 |
| final product (%) | | | | | | | |

Different enzyme concentrations were successfully tested, between 0.1% and 3%, with reaction time between 30 minutes and 10 hours. The best outcome was achieved with 0.5-1% of enzyme for 2-4 hours.

To test the pH effect on the enzymatic step, experiments were performed at different pH. The results are presented in Table 7.

TABLE 7
pH effect on the enzymatic step

| | | |
|---|---|---|
| pH | 4.5-5 | 5.5-6.5 |
| (with 2 enzyme combo - a + d) | | |
| Protein in the final product (%) | 65-75 | 55-65 |

The process showed to be efficient on different sources of Saccharomyces cerevisiae. The optimized process was run on different streams and the resulting protein content is presented in Table 8.

TABLE 8
Protein content obtained from different sources of saccharomyces cerevisiae

| | Live | Beer | Beer | Yeast | Yeast | | |
|---|---|---|---|---|---|---|---|
| | baker's | brewing | brewing | extract | extract | Autolyzed | Autolyzed |
| Raw material | yeast | stream 1 | stream 2 | stream 1 | stream 2 | yeast 1 | yeast 2 |
| Protein in the | 50-60 | 58-65 | 65-70 | 70-80 | 65-75 | 60-70 | 65-75 |
| final product (%) | | | | | | | |

The Functional properties of the final products, such as particle size, protein fractions size, emulsification and water and oil holding capacities, were analyzed.

### Particle Size

The particle size distributions of samples collected after spray drying were analyzed with a Malvern particle size analyzer (Table 9).

TABLE 9
Particle size distribution

| Drying fraction | D10 (μm) | D50 (μm) | D90 (μm) |
|---|---|---|---|
| cyclone | 5.12 | 15.3 | 40.6 |
| collector | 17.9 | 35.3 | 81.8 |

### Protein's Size—SDSpage Analysis

SDS-PAGE analysis was performed to separate the different proteins and protein subunits of the sample in function of their molecular weights. Analysis was performed in duplicate.

Some faint protein bands were observed around 80 kDa, 60 kDa, 45 kDa, 35 kDa, 30 kDa, 27 kda and 25 kDa. Small peptides are also visible below 10 kDa, as well as insoluble protein residues.

### Emulsification

The emulsifying properties of the sample were tested at pH 7 by producing oil in water emulsions in standard conditions and measuring the size distributions of the oil droplets by particle size distribution. A good emulsifier is able to form an emulsion with small droplets stable over time. Caseinate was used as a reference for this test.

Caseinate produced small oil droplets (<5 m) stable with time and can be considered very good emulsifier. Yeast extract produced a stable emulsion, but it was formed of larger oil droplets (75 m). The oil droplet size was reduced to 25 m with higher protein content (5%) (Table 10). The sample proved to be a good emulsifier.

TABLE 10
Particle size distribution

| Emulsifying properties | D50 in | D50 in | Flocculation |
|---|---|---|---|
| of samples | water (μm) | SDS (μm) | index a |
| Sodium caseinate | 1.61 | 1.63 | 1 |
| 1% protein | | | |
| Yeast extract | 75.3 | 45.1 | 0.60 |
| 1% protein | | | |
| Yeast extract | 25.6 | 5.84 | 0.23 |
| 5% protein | | | |

### Production of a Solid Emulsion

A solid emulsion was produced at pH 7 at a water:oil:protein ratio of 100:338:5. The product was semi-solid, similar to a regular mayonnaise.

The viscosity of the solid emulsion obtained was analyzed with a rheometer immediately after production and after storage. Sample presented a shear-thinning property (reduction of viscosity with shear rate), which is characteristic of structured fluids. The viscosity of the emulsion was relatively high. The viscosity did not evolve significantly after 3 days of storage.

Table 11 presents the water (WHC) and oil (OHC) holding capacity of the final yeast extract protein powder compared to soy and whey.

TABLE 11
Water (WHC) and oil (OHC) holding capacity

| | WHC | OHC |
|---|---|---|
| Yeast extract | 1.7-2.8 | 1.1-1.5 |
| protein powder | | |
| Soy | 2.7 | 0.7 |
| Whey | | 1.86 |

### Nutritional Values

Table 12 summarizes nutrition values information of a final product obtained from yeast.

TABLE 12
Nutritional analysis (Expected values for 100 g of final product)

| | |
|---|---|
| Protein | ≥65% |
| Protein | 0.94 |
| quality - | |
| PDCAAS | |
| Moisture | ≤7% |
| Energy | 412 kcal |
| Ash | 3.95 |
| Fat | 7.1 |
| Which of is | 1.4 |
| saturated | |
| Which of is | 5.7 |
| unsaturated | |
| Cholesterol | 0 |
| Carbohydrates | 17 |
| Dietary fiber | 7.6 |
| Sugars | 2.2 |

The amino acid profile was evaluated (Table 13).

TABLE 13
Amino acid profile (relative proportion of
essential amino acid per 100 g of protein)

| Amino acid | |
|---|---|
| Tryptophane | 1.05 |
| Threonine | 3.34 |
| Aspartic acid | 7.24 |
| Serine | 3.38 |
| Lysine | 5.66 |
| Valine | 4.11 |
| Proline | 2.6 |
| Alanine | 3.89 |
| Phenylalanine | 3.29 |
| Isoleucine | 3.54 |
| Glycine | 3.09 |
| Tyrosine | 2.71 |
| arginine | 3.47 |
| Leucine | 5.44 |
| Histidine | 1.68 |
| Glutamic acid | 8.25 |
| Methionine | 1.17 |
| Cysteine + | 0.6 |
| Cystine | |

### Protein Solubility

Protein solubility was measured under acidic, neutral and alkaline conditions (Table 14). The tested sample showed low protein solubility under all conditions.

TABLE 14
Protein solubility

| pH | Protein solubility |
|---|---|
| 3.5 | 7% |
| 7 | 13% |
| 9 | 20% |

The final products were stable at least for 2 years when stored at ambient temperature in dry conditions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for optimizing a protein content extracted from a microorganism comprising a cell wall, the method comprising:
a) breaking the cell wall of said microorganism to a particle size suitable for enzymatic reaction; and
b) receiving a first fraction comprising soluble proteins and a second fraction comprising insoluble proteins, wherein any one of said first and said second fraction comprises a protein content between 10% and 90% by weight of said fraction,
thereby optimizing the protein content extracted from the microorganism comprising a cell wall.

2. The method of claim 1, wherein said particle size suitable for enzymatic reaction is between 2 μm and 10 μm.

3. The method of claim 1, wherein said breaking the cell wall comprises applying any one of a) pressure between 150 bars and 1500 bars; b) temperature between 50° C. and 100° C.; c) ultrasonication, d) bead mill homogenization, or any combination thereof to said microorganism.

4. The method of claim 1, wherein said first fraction, said second fraction, or both is suitable for extrusion.

5. The method of claim 1, wherein said microorganism is a bacteria, a fungi or a microalgae, and optionally wherein said fungi is a yeast; and optionally wherein said yeast comprises Saccharomyces cerevisiae, Streptomyces natalensis, Streptomyces chattanoogensis, Saccharomyces fragilis, Candida utilis, Candida guilliermondii, Candida lipolytica, Cyberlindnera jadinii, Pichia pastoris, or any combination thereof; said yeast is derived from downstream food-related industries; said yeast comprises spent yeast, instant yeast, inactive yeast, live yeast, or any combination thereof, or any combination thereof.

6.-9. (canceled)

10. The method of claim 1, wherein any one of said first fraction and second fraction comprises a particle characterized by an average particle size between 1 μm and 100 μm.

11. The method of claim 1, wherein said protein is characterized by a molecular weight between 1 kDa and 250 kDa.

12. The method of claim 1, wherein said insoluble protein is characterized by an aqueous solubility of less than 300 g/L.

13. The method of claim 1, wherein said applying pressure comprises high-pressure homogenization.

14. The method of claim 1, further comprising a step comprising removing a fiber from said cell wall or portion thereof, optionally wherein said removing a fiber comprises chemical hydrolysis, enzymatic hydrolysis, or both, optionally wherein said enzymatic hydrolysis comprises contacting said microorganism with an enzyme, wherein said enzyme is selected from the group consisting of: lipase, phospolipase, polygalacturonase, chitinase, pectinase, β-glucanase, protease, arabinanase, pectinase, endo-amalyase, endo-β-glycanase, hydrolase, cellulose, arabanase, cellulase, hemicellulase, xylanase, laccase, mannanase, exo-endo-protease, or any combination thereof, optionally wherein said enzyme comprises phospolipase, polygalacturonase, chitinase, pectinase, β-glucanase, protease and arabinanase, or any combination thereof.

15.-17. (canceled)

18. The method of claim 14, wherein said enzymatic hydrolysis is performed at a pH between 3.5 and 7.

19. The method of claim 3, comprising repeating said applying pressure between 150 bars and 1500 bars.

20. The method of claim 1, further comprising a step comprising cleaning, grinding, milling, washing, drying or any combination thereof, said microorganism.

21. The method of claim 1, wherein any one of said first fraction and said second fraction is in the form of a powder, solution, suspension, or any combination thereof.

22. The method of claim 1, further comprising a step comprising isolating, purifying, concentrating, or any combination thereof, a protein from said first fraction, said second fraction or both.

23. A composition comprising a first fraction, a second fraction or both, derived from a microorganism comprising a cell wall and comprising a protein content between 10% and 90% by weight of said fraction, obtained by the method of claim 1.

24. A composition comprising more than 50% by dry weight of a protein derived from a microorganism comprising a cell wall, and optionally wherein said protein is characterized by a molecular weight between 1 kDa and 250 kDa, wherein said composition being characterized by an aqueous solubility of less than 300 g/L, or both.

25.-26. (canceled)

27. The composition of claim 23, wherein any one of: (i) said protein is characterized by an amino acid profile presented in Table 13 ii said composition is in the form of a powder characterized by an average particle size between 1 μm and 100 μm; (iii) said composition comprises a fiber content between 5% and 20% by dry weight; (iv) said composition comprises an ash content of less than 5%; (v) said composition is characterized by a protein digestibility-corrected amino acid score (PDCAAS) between 0.7 and 1; (vi) said microorganism is a bacteria, a fungi or a microalgae, and optionally said fungi is a yeast; and (vii) any combination of (i) to (vi).

28.-33. (canceled)

34. The composition of claim 27, wherein any one of: (i) said yeast comprises Saccharomyces cerevisiae, Streptomyces natalensis, Streptomyces chattanoogensis, Saccharomyces fragilis, Candida utilis, Candida guilliermondii, Candida lipolityca, Cyberlindnera jadinii, Pichia pastoris, or any combination thereof; (ii) said yeast is derived from downstream food-related industries; (iii) said yeast comprises spent yeast, instant yeast, inactive yeast, live yeast, or any combination thereof; and (iv) any combination of (i) to (iii).

35.-36. (canceled)

37. A food product comprising the composition of claim 23, and optionally wherein said food product is characterized as being suitable for use as an equivalent product to meat, eggs, cheese, dairy products, meat substitute products, plant-based products, or any combination thereof.

38. (canceled)
